(19) United States
(12) Patent Application Publication
(10) Pub. No.: US 2026/0354407 A1
(43) Pub. Date: Nov. 12, 2026

(54) Reduction of viral disease transmission by avian antibodies

(71) Applicant: Camas Incorporated, Le Center, MN (US)

(72) Inventors: Scott Simplot, Boise, ID (US); Bradley M. Mitteness, Ghent, MN (US); Michelle Hawkins, Bloomington, MN (US); Jason Hull, Wyoming, MN (US); Brian Robley, Mankato, MN (US); Philip Larson, Mankato, MN (US); Michael Brielmaier, Mankato, MN (US); Benjamin Willaert, Mankato, MN (US); Vergene Harkins, Le Center, MN (US)

(21) Appl. No.: 19/364,838

(22) Filed: Oct. 21, 2025

Related U.S. Application Data
(63) Continuation of application No. 17/313,738, filed on May 6, 2021, now abandoned.
(60) Provisional application No. 63/022,251, filed on May 8, 2020.

(51) Int. Cl. A23G 3/36; A61K 35/57; C07K 16/104; C07K 16/12; C07K 16/14; C07K 16/20

(52) U.S. Cl. CPC A23G 3/364; A61K 35/57; C07K 16/104; C07K 16/12; C07K 16/14; C07K 16/20; C07K 2317/76

(57) ABSTRACT

Formulations of egg powders with avian antibodies that act as mucosal protectants are provided. The formulations are effective for protecting the mucosal membranes in the pharyngeal area of an individual from viral infections. Formulations effective for reducing infections of SARS-CoV-2 in individuals are provided. Formulations are also effective for reducing transmission of viral disease. The formulations are tablets, candies, gummies, throat lozenges, and powders for administering to the pharyngeal area. Methods for administering the formulations for reducing viral disease such as Covid-19 are disclosed.

### CROSS-REFERENCE TO RELATED APPLICATION

The present application is a continuation of U.S. patent application Ser. No. 17/313,738, filed May 6, 2021, which is based on and claims priority to U.S. provisional patent application Ser. No. 63/022,251, filed May 8, 2020, the contents of which are hereby incorporated by reference in their entirety.

### FIELD

The present description relates to delivery of antibodies and more particularly to delivery of avian antibodies to reduce viral disease transmission.

### BACKGROUND

Severe acute respiratory syndrome coronavirus-2 (SARS-CoV-2) is the virus strain that causes coronavirus disease 2019 (CoVID-19), a respiratory illness. SARS-CoV-2 is a positive-sense single-stranded RNA virus.

Transmission of SARS-CoV-2 can occur by human-to-human transmission and thus humans in constrained spaces can lead to increase in transmission rates. Transmission occurs primarily via respiratory droplets from coughs and sneezes within a range of about 1.8 meters (6 ft). The virus may also be transmitted as aerosol particles. Indirect contact via a contaminated surface is another possible cause of infection. Preliminary research indicates that the virus may remain viable on plastic and steel for up to three days, but does not survive on cardboard for more than one day or on copper for more than four hours; the virus is inactivated by soap, which destabilizes its lipid bilayer.

### SUMMARY

In one aspect, the present description relates to a composition for reducing respiratory disease. The composition can include a formulation having egg material. The egg material may be a powder or a liquid having one or more avian antibodies. The egg material is formulated for dispersion onto the mucous membranes of the pharyngeal area in an individual. The one or more avian antibodies in the formulation may bind and/or neutralize one or more viruses. The egg material may be produced from eggs laid by female birds, wherein the birds are chickens inoculated with one or more viruses or viral antigens causing the respiratory disease. The formulation may be an oral tablet, a candy, a gummy or a throat lozenge. The formulation may be a powdered nasal inhalant or a nasal spray. The one or more virus may be Influenza viruses, Coronaviruses, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus, West Nile virus, human respiratory syncytial virus (RSV), human metapneumovirus (hMPV), Picornaviridae, rhinoviruses, enteroviruses and combinations thereof. The one or more virus may be from the family Coronaviridae. The one or more virus may be severe acute respiratory syndrome coronavirus-2 (SARS-CoV-2). The avian antibodies may be directed against an antigen from SARS-CoV-2, wherein the antigen includes s2 spike protein, s1 spike protein, spike rbd protein, nucleocapsid, envelope or combinations thereof. The formulation may further include avian antibodies against disease-causing organisms that cause a secondary infection, wherein the disease-causing organisms include bacteria, fungi, protozoa, worms or combinations thereof. The formulation may further include excipients. The excipients may include binders, coatings, disintegrants, flavors, lubricants, preservatives, sorbents, sweeteners, or combinations thereof. The egg material may be whole egg material, partially purified egg material, purified avian antibodies or combinations thereof.

In a further aspect, the present description relates to a method of delivering avian antibodies to an individual. The method includes administering a formulation that includes egg material. The egg material may be a powder or a liquid. The egg material can include one or more avian antibodies specific for one or more target viruses. The egg material is formulated to coat all or a portion of mucosal surfaces in the individual's pharyngeal area. The one or more avian antibodies in the formulation binds and/or neutralizes the one or more target viruses. The formulation may include an oral tablet, a candy, a gummy or a throat lozenge. The formulation may include a powdered nasal inhalant or a liquid nasal spray. The avian antibody formulation may prevent a viral fusion molecule in the one or more viruses from attaching to the cellular attachment site of cells in the pharyngeal area of an individual. The avian antibody formulation may reduce the transmission of disease by binding and neutralizing the one or more viruses in the pharyngeal area of the individual. The individual may be uninfected and the neutralizing prevents the one or more viruses from infecting the individual. The individual may be infected and the neutralizing prevents the one or more viruses from infecting a second individual, wherein the second individual in an uninfected individual. The formulation may be administered to the nasopharyngeal area. The formulation may be administered to the oropharyngeal area. The formulation may be placed in the mouth and chewed to release the avian antibody. The formulation may be placed in the mouth and held under the tongue or inside the upper lip for slow release of the avian antibodies from the formulation. The formulation may be held in the mouth for at least 2 minutes, or at least 10 minutes, or at least 20 minutes, or at least 30 minutes. The one or more virus may include Influenza viruses, Coronaviruses, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus, West Nile virus, human respiratory syncytial virus (RSV), human metapneumovirus (hMPV), Picornaviridae, rhinoviruses, enteroviruses and combinations thereof. The one or more virus may include the family Coronaviridae. The one or more virus may be SARS-CoV-2. Administering the formulation may prevent Covid-19. The avian antibody may bind Spike Glycoprotein S. The cellular attachment site in the cells may be the ACE2 receptor in the individual. The avian antibodies may be directed against an antigen from SARS-CoV-2. The SARS-CoV-2 antigen may be s2 spike protein, s1 spike protein, spike rbd protein, nucleocapsid, envelope or combinations thereof. The formulation may further include avian antibodies specific against bacteria, fungi, protozoa, worms or combinations thereof.

In yet another aspect, the present description relates to a method of reducing transmission of a respiratory disease. The method includes administering a formulation including egg material, wherein the egg material includes one or more avian antibodies to coat all or a portion of mucosal surfaces in the individual's pharyngeal area. The formulation may bind and neutralize one or more target viruses causing the respiratory disease. The formulation may include avian antibodies specific for the one or more viruses. The method may reduce disease transmission from an infected individual to an uninfected individual, wherein the uninfected individual is administered the avian antibody formulation. The method may reduce disease transmission from an infected individual to an uninfected individual, wherein the infected individual is administered the avian antibody formulation.

### BRIEF DESCRIPTION OF THE DRAWINGS

FIG. 1A is a graph depicting the absolute neutralization of virus observed at 1:10 dilution of the antibodies.

FIG. 1B is a graph depicting the same data as FIG. 1A as relative percentage of neutralization observed in the context of the four antibodies as compared to the control.

### DEFINITIONS

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers to one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The term “and/or” means one or all of the listed elements or a combination of any two or more of the listed elements.

The terms “comprises,” “comprising,” and variations thereof are to be construed as open ended—i.e., additional elements or steps are optional and may or may not be present.

The term “microbial adherence inhibitor” as referred to herein includes molecules that bind and inhibit microbes, e.g. bacteria, viruses and the like.

The term “antibody” as referred to herein relates to immunoglobulin molecules and is one embodiment of a microbial adherence inhibitor. The embodiments are described herein with reference to antibodies but it will be understood that microbial adherence antibodies other than antibodies may also be used and all are within the scope of this description.

The term “disease-causing organisms” as referred to herein includes viruses, bacteria, fungi, protozoa, worms and combinations thereof.

The term “immunogens” as referred to herein includes disease-causing organisms and/or material derived from disease-causing organisms that are used as antigens for inoculating hens.

The term “pharyngeal area” as referred to herein includes the oropharynx, nasopharynx, and laryngopharynx of an individual.

The term “virus fusion molecule” or “viral fusion molecule” as referred to herein relates to the molecule or molecules on a virus that are involved in binding and/or attaching to the cells of the individual host.

The term “cellular attachment site” as referred to herein relates to a molecule, e.g. a receptor, on the host cells that is the site for viral attachment.

The term “neutralize” as referred to herein relates to binding by avian antibodies to a disease-causing organism, e.g. virus, and prevents the attachment of the disease-causing organism to the cellular attachment site in the cells of an individual.

The term “secondary infections” as referred to herein relates to infections caused by a disease-causing organism after a first disease-causing organism, e.g. a virus, has infected an individual. The individual can become susceptible to the secondary infection due to a weakened immune system as a result of the primary or first infection.

The term “spike protein” as referred to herein relates to whole spike protein and/or portions of spike protein, e.g. the S1 subunit, the S2 subunit, portions of the S1 and S2 subunits.

The term “anti-spike protein antibodies” as referred to herein relates to antibodies directed to the whole spike protein or portions of the spike protein, e.g. the S1 subunit, the S2 subunit, portions of the S1 and S2 subunits. The anti-spike protein antibodies includes anti-S1 spike antibodies, anti-S2 spike antibodies and the like.

The term “antibody product” as referred to herein relates to formulations that includes the antigen specific IgY antibodies and can include, for example, candies, tablets, gummies and the like. The products/formulations can be oral and/or nasal formulations.

The term “oral formulation” as referred to herein relates to a tablet, a candy, a gummy, a throat lozenge or other formulations that can be administered orally to an individual.

### DETAILED DESCRIPTION

The present description includes formulations that include microbial adherence inhibitors, in the form of fowl egg antibodies. The microbial adherence inhibitors, e.g. antibodies, are formulated for delivery to coat and protect the mucous membranes of the oropharyngeal and/or nasopharyngeal areas in individuals. The formulations can bind and/or neutralize viruses in the pharyngeal area of an individual.

The formulations can be directed to substantially prevent the attachment or adherence of disease-causing organisms, e.g. viruses, by inhibiting the ability of the organism to adhere to the mucous membranes of individuals and cause respiratory disease. The formulations can reduce disease transmission by preventing entry of viruses into an uninfected individual. The formulations can also reduce disease transmission by reducing infectious particles that are expelled from an infected individual. The present description includes formulations of avian antibodies that are delivered easily and economically to individuals susceptible to viral infections and to individuals harboring viral infections. The antibodies in the formulations may be slowly released.

In some embodiments, the formulations are oral formulations such as a tablet, a throat lozenge, a candy, a chewable/gummy product and the like. The oral formulations can coat and protect the oropharyngeal mucous membranes and prevent entry of the virus into the individual to reduce the risk of infection. In some embodiments, the formulations are nasal formulations. In some embodiments, the nasal formulations are powdered formulations of avian egg antibodies for nasal administration by inhalation through the nasal passages to coat and protect the nasopharyngeal mucous membranes and prevent the risk of infection. The present description also includes methods of preventing or reducing the infection of an individual by one or more viruses. The present description also includes methods of reducing transmission of viral disease by decreasing infectious viral particles expelled by an infected individual. The methods can include administering formulations of avian egg antibodies to coat and protect the pharyngeal mucous membranes in order to neutralize the one or more viruses that cause respiratory disease.

In many viral diseases, the spread of the virus is greatest during the asymptomatic phase or the early symptomatic phase that is around and immediately following the onset of symptoms. Often individuals can be exposed to respiratory viruses and risk being infected by being in the presence of infected individuals that are asymptomatic or have mild and/or non-specific symptoms.

Widespread outbreaks of viral infections can have significant health and economic costs. The ability to prevent and/or reduce the transmission of viral infections, especially using low-cost therapies that are easily administered, can be advantageous in developing countries as well as the whole world. In some embodiments, the present description can include formulations of a mucosal protectant that can be delivered easily and at low-cost. The formulations described herein can be stable for use in a variety of settings. The formulations can be widely distributed to individuals and stored until use. In some embodiments, they can be self-administered, as needed.

Exposure to respiratory viruses such as SARS-CoV-2 can lead to respiratory infections as a result of the entry of the virus through the mucous membranes in the pharyngeal areas when an individual is exposed to the virus. Without any mucosal protectant, the viruses can make the journey from the pharyngeal area to the lower respiratory tract to the interior of the lungs. This can lead to the respiratory diseases such as Covid-19. Transmission between humans can be through expelled/discharged viral particles in respiratory droplets, saliva, airborne droplets and the like. Once the microorganisms are established in the pharyngeal area, respiration can result in downward carriage of the viral pathogens into the lower respiratory tract. This allows the organisms to attach to the bronchi and alveolar cells and to multiply and increase viral load.

The formulation products described herein can be all natural preparations that contain specific avian antibodies to the targeted immunogens. These antibodies when attached to the outer surface cell walls can prevent the organism from attaching to the cells in the mucous membranes. The microorganisms will not be able to multiply or colonize in the individual upon binding by the specific avian antibodies. The formulation can keep the microorganisms from moving down the respiratory tract and eliminate the ability to cause disease in the lower respiratory tract. By coating the antibody material on mucous membranes of the pharyngeal area, the coating can prevent the viruses or other microorganisms from infecting an individual. Delivery of the formulations described herein provide for a substantial decrease in infections in individuals and transmission of the disease caused by the viruses.

Viruses have evolved a number of different types of molecules, referred to herein as viral fusion molecules, on their surfaces which can very tightly stick to one or more types of specific molecules that are part of the host's cell surfaces. The avian antibodies described herein can be of extraordinary high specific activity which can very tightly bind to, coat, cover and obliterate these viral fusion molecules which attach themselves to their host cell's attachment sites with a lock and key type of fit to very unique chemical structures. The avian IgY immunoglobulins in the yolk tightly bind to, coat, cover and obliterate viral fusion molecules to prevent the attachment of viruses to their hosts.

The formulations described herein can be used as a mucosal protectant of the pharyngeal area against one or more viruses in individuals. In some embodiments, the individuals may be susceptible due to potential exposure to an infected individual(s) in a workplace, home, school, or other public or private settings. In some embodiments, the individual may be immunosuppressed and is more susceptible to viral infections when exposed to an infected individual(s). In some embodiments, the individual may use the formulations for prophylactic purposes due to prolonged exposure to individuals with an unknown infection status. In some embodiments, the individual may be an infected individual and use of the formulation can reduce the number of infectious particles that are discharged by the infected individual into the environment.

In some embodiments, the formulations can include microbial adherence inhibitors, e.g. antibodies. The embodiments described herein will be discussed in the context of avian antibodies, but it will be understood that other microbial adherence inhibitors may be used and are within the scope of this description. In some embodiments, the avian antibodies are derived from birds inoculated with the target immunogens, e.g. viral antigens, bacterial antigens and the like. Birds, in particular, have the ability to “load up” their eggs as they are formed, with a very large supply of antibodies. Once immunized, the hen deposits IgY type immunoglobulins in the yolk while depositing IgM and IgA immunoglobulins in the albumin. The albumin helps add stability to the whole egg preparations and helps protect the avian antibodies. The avian IgY immunoglobulins in the yolk can tightly bind to, coat and cover the mucosal membranes in an individual to bind and inhibit the entry of the target virus particles. The IgM and IgA immunoglobulins in the albumin increase binding of the antibody containing material in the mucous membrane in the pharyngeal area. This can provide longer sustaining effect of the antibody containing material. The larger antibody containing molecules are more effective in preventing adherence of the targeted immunogen, e.g. virus, in the pharyngeal area of the human. Albumin is a protein that protects the activity of the IgY immunoglobulins thereby increasing their active life. Furthermore, a large fraction of the antibodies deposited in the eggs by the hen are against the most recent antigenic challenges to the hen. This all results in the eggs of birds being a most ideal source for large quantities of economically produced highly specific and stable antibodies. While the present description is illustrated by the use of chickens to produce avian antibody, other fowl including turkeys, ducks, geese, ostrich, Emu, pheasant, pigeon, quail, etc. or combination thereof, may be used.

In addition to this direct attack by administered antibodies, components of the complement system included in most biological fluids, such as blood, lymph, saliva, tears and to some extent intestinal secretions, recognize an antibody attachment as triggers for their many types of defensive activities. Specific antibody attachment and coating combined with the very likely mobilization of many other cellular defense systems, therefore, quickly culminating in the chemical inactivation and ultimately the destruction of the targeted microorganism.

The avian antibodies in the formulations can be specific for the target immunogens. The target immunogens can be viral antigens from one or more target viruses that can be injected into the hens to generate the avian antibodies. The viral antigens can be virus particles, live or inactivated, viral proteins, viral macromolecules, viral fragments, and/or any other material derived from viruses. In one embodiment, virus particles can be used as immunogens to inoculate hens in order to generate the desired avian antibodies for formulation. In one embodiment, the immunogen can include all or part of a protein that can be present on the surface of the viral particles. In one embodiment, the immunogen can be all or part of the molecule that can bind and/or attach to the cellular attachment site in the individual's cells. In one embodiment, the immunogen can be the viral fusion molecule, e.g. the virus molecule that attaches to an individual's target cell.

In some embodiments, the viral antigens inoculated into the chickens are isolated and/or derived from viral particles. In some embodiments, the viral antigens used as immunogens are recombinant molecules. In some embodiments, the viral antigens, e.g. polypeptides, are expressed in recombinant expression systems. The viral antigens may be isolated from the recombinant expression systems and used as immunogens for inoculating the chickens.

In some embodiments, the viral fusion molecule can include all or part of a polypeptide fragment that is expressed on the surface of the viral particles. In one embodiment, the viral fusion molecule can be the Spike Glycoprotein S protein in SARS-CoV-2. In some embodiments, the viral antigens can include antigens from SARS-CoV-2 such as s2 spike protein, s1 spike protein, spike rbd protein, nucleocapsid, envelope and/or combinations thereof. In some embodiments, the antigens from SARS-CoV-2 can include nucleocapsid protein (GTX135357-pro), Spike RBD protein, His tag (active) (GTX01546-pro), Envelope protein, His and Avi tag (GTX01547-pro). These antigens can be purchased from, for example, GeneTex, Irvine, CA. Other membrane proteins that are immunogenic and can inhibit the binding of a viral particle to the target cells in an individual may also be used and all are within the scope of this description. In one embodiment, the viral fusion protein can also include other molecules such as sugars, lipids and the like. In one embodiment, inactivated SARS-CoV2 viral particles may be used as immunogens.

In some embodiments, groups of young hen chickens can be obtained such as Rhode Island Reds, White Leghorns, sex-linked hybrid crosses or other breeds suited to large egg size, high volume egg production and ease of handling which are about to reach laying age, about 16-19 weeks for chickens, on a schedule predetermined by the amount and timing of final product desired resulting in a steady continuous production stream. After a suitable period of isolation and acclimatization of about two to four weeks, each group can enter into an inoculation program using preparations of specific antigens (immunogens) such as viruses, to which an antibody is desired. In some embodiments, the cultures of microorganisms may be obtained from commercial sources such as the American Type Culture Collection (ATCC) or from wild type isolates. In some embodiments, the microorganisms may be cultured from infected individuals. The cultures may be used to isolate antigens.

The viral antigens can be prepared as immunogens and the hens may be injected intramuscularly, but preferably injected subcutaneously. In approximately four to five weeks, the average egg collected will contain copious amounts of the desired specific antibody in a readily usable and stable form. The chickens may be re-inoculated with the targeted immunogen throughout the egg laying period to maintain the high antibody level.

In some embodiments, variants of the SARS-CoV2 virus or antigens for variants of SARS-CoV2 may be used to inoculate the birds. Viruses evolve over time and variants of a virus can emerge as the virus circulates in the world. A variant can have one or more mutations in the amino acid sequences of the proteins relative to the original viral particles. Variants may be more or less infectious than the original virus. In one embodiment, the immunogens used to inoculate the hens are derived from variants of the SARS-CoV2 virus. Variants can include, for example, the Brazil variant, the UK variant or the South African variant. Variants can also include, for example, B.1526, B.1526.1, B.1.525 and P.2, B.1.1.7, B.1.351, P.1, B.1.427, B.1.429 and the like. Other variants that may emerge can also be used as immunogens.

Batches of eggs from predetermined groups of chickens can be cracked, the contents can be separated from the shells and mixed and preferably pasteurized to eliminate potential pathogenic microorganism. Standard test procedures are used, such as ELISA, agglutination, or the like are used to the monitor the antibody activity. The typical batch is then blended with batches from groups of chickens at other average production levels resulting in abundant standardized active ingredients.

In embodiments for formulations specific to one target virus, the formulations can be derived from egg material wherein the hens are inoculated with viral antigens from the one target virus. In some embodiments, all of the hens may be inoculated with the same antigen from one virus. In some embodiments, the hens may be inoculated with different antigens from the same target virus and the egg material from all the hens may be combined.

In embodiments for formulations specific for more than one virus, the formulations can be derived from egg materials from a variety of hens that have been inoculated with viral antigens from different target viruses. The egg material specific for the different target viruses may be combined to form a formulation that is specific some or all of the target viruses.

In some embodiments, formulations with the avian antibodies that cross-react with the target virus may be used. In such embodiments, the avian antibodies may be specific for a first non-target virus and these avian antibody formulations may be used against a desired target virus.

In some embodiments, the egg material that includes the avian antibody material may be used without further purifications in the formulations. In some embodiments, the avian egg material may be partially purified such as, for example, by water extractions. In some embodiments, the egg material may be substantially or fully purified to obtain the avian antibodies that bind the target disease-causing organism. In one embodiment, the egg yolks may be separated from the egg whites. The egg yolk material obtained after disrupting the yolk sac may be used in the formulations described herein. The egg materials or egg yolk material may be used as a liquid or it may be further processed to form egg powder or egg yolk powder. Methods of forming powders from egg material are known in the art and any of these methods may be used to obtain the egg powders. The antibodies may be purified, dried and lyophilized for storage for later use.

Dependent on the needs and specifications of the formulator and the final customer, the final formulations may include excipients or some type of innocuous additive. In some embodiments, the formulations can include excipients. A variety of excipients are known in the field and can be included in the formulations and all are within the scope of this description. The excipients can vary depending on the nature of the formulation. Excipients can include, for example, anti-adherents, binders, coatings, colors, disintegrants, flavors, glidants, oils, lubricants, preservatives, sorbents, sweeteners, vehicles and the like. Binders can include, for example, saccharides, e.g. sucrose, lactose, starches, celluloses, xylitol, sorbitol, mannitols, gelatin, polyvinylpyrrolidone (PVP) polyethylene glycol (PEG) and the like. In some embodiments, dry binders such as cellulose, methylcellulose, PVP, PEG and the like may be added to a powder blend that includes avian antibody powder either after a wet granulation step or as part of a direct powder compression. The amounts of excipients in formulations can vary and can be dependent on the desired formulation and/or dosage.

The formulations with the avian antibodies described herein can bind and inhibit a variety of viruses. The formulations can include avian antibodies against DNA viruses and/or RNA viruses. In some embodiments, the viruses can be, for example, Influenza viruses, Coronavirus, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus and West Nile virus. In some embodiments, the formulations with the avian antibodies can be directed against viruses that include RNA viruses of the order Mononegavirales that contain single-stranded genomes that are negative sense. These viruses can include, for example, Orthomyxoviridae (which contains the influenza viruses) and Paramyxoviridae (which contains the parainfluenza viruses (PIVs), human respiratory syncytial virus (RSV), and human metapneumovirus (hMPV). The viruses inhibited by the formulations can also include, for example, Picornaviridae, rhinoviruses, enteroviruses (such as coxsackieviruses and numbered enteroviruses). In some embodiments, DNA viruses such as Adenoviridae, Parvoviridae and the like can also be inhibited.

In some embodiments, the viruses inhibited by the formulations can include, for example, viruses from the family Coronaviridae, an enveloped, positive-sense single-stranded RNA (ssRNA). These viruses can include, for example, human coronavirus (HCoV) 229E, HCoV OC43, the severe acute respiratory syndrome-associated CoV (SARS-CoV), Middle East respiratory syndrome-related coronavirus (MERS-CoV), HCoV, NL63, HCoV HKU1 and the like. In one embodiment, virus detections system in facemasks can detect SARS-CoV-1 and/or SARS-CoV-2.

In some embodiments, the avian antibodies may be directed towards antigens of influenza viruses and other microorganisms that may cause respiratory diseases in individuals. The viruses can be, for example, Orthomyxoviridae, specifically influenza, H1N1, H5N1, H3N2, or combinations thereof or other types of Hemagglutinin (H) and neuraminidase (N) combinations that are typically identified by an H number and an N number and their mutated strains; the Herpesviridae, specifically, Infectious Bovine Rhinotracheitis, 1 and 5; the Paramyxoviridae, specifically BRSV and Pls; the Arteriviridae, specifically, porcine respiratory and reproductive syndrome virus (PRRSv) and the Adenoviridae, specifically Bovine adenovirus 1, 3, 5, 6, 7.

In some embodiment, the formulations may include avian antibodies that bind and/or neutralizes other disease-causing organisms in addition to the target viruses. The susceptibility for a secondary infection by a second disease-causing organism may be increased in individuals that are fighting a primary infection by the target virus or viruses. In some embodiments, bacteria, viruses, fungi, protozoa and the like can cause a secondary infection.

In some embodiments, the avian antibodies in the formulations may be directed against disease-causing organisms that cause secondary infections. The disease-causing organisms causing secondary infections can include other viruses as described above, and bacteria, protozoa, and the like. The disease-causing organisms that can cause secondary infections can include, for example, Gram positive cocci, Gram positive rods, Gram negative cocci, Gram negative rods and the like. Gram positive cocci can include, for example, Streptococcus pneumoniae, Streptococcus pyogenes, Streptococcus agalactiae, Staphylococcus aureus, Peptostreptococcus anaerobe. Gram positive rods can include, for example, Bacillus anthracis, Nocardia, Actinomyces anaerobe. Gram negative cocci can include, for example, Neisseria meningitidis, Moraxella catarrhalis. Gram negative rods can include, for example, Klebsiella pneumoniae, Escherichia coli, Pseudomonas aeruginosa, Acinetobacter, Burkholderia pseudomallei, Burkholderia mallei, Yersinia pestis, Francisella tularensis, Hemophilus influenzae, Bordetella pertussis, Bacteroides melaninogenicus anaerobe, Fusobacterium anaerobe, Porphyromonas anaerobe, Prevotella anaerobe, Proteus, Serratia. The disease-causing organisms can also include, for example, Mycobacterium tuberculosis, other Mycobacterium, Legionella pneumophila, Mycoplasma pneumoniae, Chlamydia trachomatis, Chlamydia psittaci, Chlamydia pneumonia, TWAR agent, Coxiella burnetii.

In one embodiment, an individual susceptible to infection by SARS-CoV-2 may also be susceptible to infections by bacteria such as Streptococcus pneumoniae, Haemophilus species, Staphylococcus aureus and Mycobacterium tuberculosis. Thus, an avian antibody formulation may include avian antibodies against SARS-CoV-2, Streptococcus pneumoniae, Haemophilus species, Staphylococcus aureus and Mycobacterium tuberculosis. Secondary infections by other potential disease-causing organisms may be alleviated by inclusion of avian antibodies against disease-causing organisms that an individual may be susceptible to when infected with the target virus or viruses.

In some embodiments, the avian antibody material can be dried into a powder. In some embodiments, the antibody material can be spray-dried powder. In some embodiments, the antibody material may include carrier materials. The antibody material may be dried prior to or after the addition of carrier materials.

Spray drying of antibody material can maintain the function of the antibodies and the ability of the antibodies to coat and protect the mucosal membranes from binding and attachment of the viral particles. Spray drying methods are known in the art and a variety of spray drying techniques may be used and are within the scope of this description. Spray drying can include atomizing of a liquid from a nozzle into a high temperature environment. The high temperature can remove the liquid in the sample. In some embodiments, the outlet air temperature for spray drying the egg material can be at a temperature below about 180 °F. In one embodiment, the outlet air temperature in the spray dryer can be between about 150 °F and about 175 °F. The outlet air temperature may be below about 175 °F, or about 165 °F.

In some embodiments, the dried egg material with the avian antibodies can include powder particles. The size of the powder particles of avian antibodies can vary and all are within the scope of this description. In some embodiments, the powder particles can include particles between about 5 microns and about 400 microns. Particles outside of this range are also within the scope of this description.

In some embodiments, the avian antibody material can be a liquid. The liquid may include whole egg material, egg yolk material, partially purified egg material and/or purified egg material. Partially purified egg material can include, for example, a polyethylene glycol extracted egg material or a water extraction egg material.

In some embodiments, the powder and/or the liquid with the avian antibodies may be formulated into an oral formulation. In some embodiments, oral formulations can include a tablet, a candy, a chewable gummy, a throat lozenge, a gargle, a mouth rinse and the like. In some embodiments, impregnated strips with the avian antibody material can be designed to dissolve in the mouth to release antibodies and can be used to coat the upper airways with the formulation.

In one embodiment, the powder and/or liquid with the avian antibodies can be formulated into a tablet. Formulation into tablets or throat lozenges are known in the art and can include the use of carrier materials and/or excipients as disclosed above. Avian antibodies and excipients, if used, can be combined and pressed or formed into tablets. The tablets may be coated or uncoated. The tablets can be formulated to be placed in the mouth and to dissolve slowly in the mouth to coat the oropharyngeal area of an individual to act as a mucosal protectant.

In some embodiments, the antibody products may also be formulated to be chewable, e.g. a gummy. Chewables can also release the avian antibody in the oral cavity and coat the oropharyngeal area of an individual to act as a mucosal protectant. The chewables may or may not be formulated for swallowing in order to enter the digestive tract. In one embodiment, formulations can include a wet granulation step. In one embodiment, formulations can include direct powder compression. In some embodiments, the avian antibody powder can be formulated to form a throat lozenge, a candy, a gummy, a tablet and may include other ingredients to lubricate and soothe irritated tissues in the mouth.

The number of doses administered per day of the oral formulations can vary and all are within the scope of this description. The formulations described herein are safe at any amounts since they are food product. There are no issues with overdosing on the antibody products described herein. In one embodiment, at least about one dose per day of the oral formulation is administered. In some embodiments, at least about two doses per day, or at least about 3 doses per day, or at least about 4 doses per day, or at least about 5 doses per day, or at least about 6 doses per day or more may be administered. Doses may be administered continually and/or sequentially. Doses may be administered if the individual is susceptible to high risk of exposure to the viruses. Doses can be administered for any length of time. In some embodiments, the doses can be administered as long as the threat of a viral infection is present. Doses may be administered for about 1-7 days, or about 7 to 14 days, or about 14 to 21 days, or about 21-31 days, or about more than one month, or about more than two months, or about more than three months, or about more than 6 months, or about more than a year.

In some embodiments, the present description can include a nasal formulation of powder particles of the avian antibody materials to coat the nasopharyngeal area of an individual as a mucosal protectant. Nasal formulations can include a nasal powder, a nasal spray and the like. In some embodiment, the nasal formulation may be powder particles that may be administered by inhalation into the nasal passages to protect the mucous membranes of the nasopharyngeal area in an individual. In one embodiment, a straw may be used to inhale a powdered nasal formulation. The powdered particle formulation may be inhaled into both of the nasal passages of the individual, separately or simultaneously. The powder particles may include excipients. The excipients may be added prior to forming, or added as a powder, after the formation of avian antibody powder material. In some embodiments, the nasal formulation may be a nasal spray that is administered by, for example, squirting an amount of nasal spray into each of the nostrils.

The number of doses administered per day of the nasal formulation, e.g. powdered avian antibody material, can vary and all are within the scope of this description. In one embodiment, at least one dose per day of the powdered formulation is administered. In one embodiment, at least two doses per day, or at least 3 doses per day, or at least 4 doses per day, or at least 5 doses per day, or at least 6 doses per day or more may be administered. Doses can be administered for any length of time. In some embodiments, the doses can be administered as long as the threat of a viral infection is present. Doses may be administered for about 1-7 days, or about 7 to 14 days, or about 14 to 21 days, or about 21-31 days, or about more than one month, or about more than two months, or about more than three months, or about more than 6 months, or about more than a year.

The amount of dried egg material in each dose can vary and all are within the scope of this description. The amount of egg powder in a formulation can depend on the type of formulation, the excipients used and the like. In some embodiments, the amount of spray dried egg powder can include between about 1 mg and about 100 grams. Amounts outside of this range are also within the scope of this description.

The amount of antibody material in each piece of an antibody product can vary and all are within the scope of this description. By each piece, it is meant a tablet, a gummy, a candy or a unit used by an individual. It will be understood that an individual may be administered one or more pieces at the same time or sequentially as described herein.

In some embodiments, each piece of an antibody product or formulation can have at least about 0.01 grams, or at least about 0.1 grams, or at least about 0.2 grams, or at least about 0.3 grams, or at least about 0.4 grams, or at least about 0.5 grams, or at least about 0.75 grams or at least about 1 gram of egg yolk powder. In some embodiments, each piece of an antibody product can have less than about 5 grams, or less than about 3 grams, or less than about 1 gram, or less than about 0.75 grams, or less than about 0.5 grams, or less than about 0.4 grams, or less than about 0.3 grams, or less than about 0.2 grams, or less than about 0.1 grams.

In some embodiments, the formulation in each piece can have, for example, between about 0.1 and 5.0 grams, or between about 0.2 and about 1.0 grams, or between about 0.3 and about 0.7 grams of egg yolk powder.

In some embodiments, the amount of egg material in the formulations may be between about 0.1 grams and about 5 grams. In some gargle embodiments, the amount of egg material may be between about 1 gram and about 25 grams. In some nasal embodiments, the amount of egg material may be between about 1 mg and about 1 gram.

In some embodiments, the formulations can include egg yolk powders that are at least about 1% by weight, or at least about 5% by weight, or at least about 10% by weight, or at least about 15% by weight, or at least about 20% by weight, or at least about 40% by weight or the whole formulation. In some embodiments, the formulations can include egg yolk powders that are less than about 75% by weight, or less than about 50% by weight, or less than about 40% by weight, or less than about 30% by weight, or less than about 20% by weight, or less than about 10% by weight. In some embodiments, the formulations can include egg powder that is between about 1% by weight and about 90% by weight, or between about 5% by weight and about 70% by weight, or between about 10% by weight and about 50% by weight, or between about 10% by weight and about 30% by weight of the formulation.

The number of specific IgY antibody molecules in the formulation of each piece, e.g. tablet, candy, gummy, can vary and all are within the scope of this description. The number of specific antibody molecules can be calculated as shown in the Examples herein. In some embodiments, the number of specific IgY antibody molecules in the formulation for each piece can be at least about 1×10⁵ molecules, or at least about 1×10¹⁰ molecules, or at least about 1×10¹² molecules, or at least about 1×10¹⁴ molecules, or at least about 1×10¹⁵ molecules, or at least about 1×10²⁰ molecules. In some embodiments, the number of specific IgY antibody molecules in the formulation of each piece can be less than about 1×10²³ molecules, or less than about 1×10²⁰ molecules, or less than about 1×10¹⁷ molecules, or less than about 1×10¹⁶ molecules, or less than about 1×10¹³ molecules, or less than about 1×10¹⁰ molecules. In some embodiments, the number of specific IgY antibody molecules in the formulation of each piece can be between about 1×10⁵ molecules and about 1×10³⁰ molecules, or between about 1×10¹⁰ molecules and about 1×10²⁰ molecules, or between about 1×10¹³ molecules and about 1×10¹⁷ molecules. In one embodiment, about 1×10¹⁵ molecules are in the formulation for each piece.

Formulations can include other components such as gelatin, sweeteners, oil, preservatives, flavors, colors and the like. Flavors can include, for example, ginger, lemon, honey, mint, strawberry, and the like. Gelatin, if used, can include, for example, between about 1% by weight and about 20% by weight of the formulation, or between about 2% by weight and about 10% by weight of the formulation. Other percentages of gelatin may be used and can be dependent on the type of formulation and all are within the scope of this description.

In some embodiments, sweeteners may be used in the formulations. Sweeteners can include, for example, honey, tapioca syrup, maple syrups, cane sugar, other syrups, sugars and the like. The amount of sweetener may vary and can be dependent on the type of formulation. The amount of sweetener can be, for example, between about 1% and about 80% of the formulation by weight, or between about 10% and about 60% of the formulation by weight, or between about 20% and about 60% of the formulation by weight. Other percentages of sweeteners may be used and can be dependent on the formulation and all are within the scope of this description.

In some embodiments, oils may be used in the formulations. Oils can include, for example, olive oil, vegetable oil, sesame oil, coconut oil, sunflower oil and the like. The amount of oil may vary and can be dependent on the type of formulation. The amount of oil can be, for example, between about 1% and about 50% of the formulation by weight, or between about 5% and about 30% of the formulation by weight, or between about 10% and about 20% of the formulation by weight. Other percentages of sweeteners may be used and can be dependent on the formulation and all are within the scope of this description.

In some embodiments, the antibody product may be placed in the mouth and chewed to release the antibody. Chewing the formulations can result in the release of the avian antibodies and coating the oropharyngeal surfaces and provide protection against the virus. Chewing can provide antibodies that can be protective within about 1 minute, or within about 2 minutes, or within about 5 minutes. In some embodiment, the avian antibodies in the formulation, when the formulation is chewed, can provide stable antibodies for at least about 5 minutes, or for at least about 10 minutes, or for at least about 15 minutes, or for at least about 20 minutes or longer.

In some embodiments, the formulations may be geared toward a slow-release of the antibodies. By slow release, it is meant that the antibodies may be released from the formulation in about 5 minutes or longer, or in about 10 minutes or longer, or in about 20 minutes or longer, or in about 30 minutes or longer, or in about 60 minutes or longer, or in about 90 minutes or longer, or in about 120 minutes or longer, or in about 4 hours or longer, or in about 8 hours or longer, or in about 12 hours or longer.

In some embodiments, the antibody may be placed in the mouth and allowed to slowly dissolve over time to slowly release the avian antibodies. In one exemplary embodiment, a candy may be placed in the mouth and held underneath the tongue and/or the upper lip. Limiting the access of the formulation from the saliva can protect the formulation from saliva proteases. The formulations may be ingested in a manner that prolongs the residence time in the mouth for a slow release of the antibodies into the mouth from the formulation. Slow release of the antibodies can prolong the protection provided by the formulation. As shown below in Examples, the IgY antibodies in the formulation are stable when slowly released. In some embodiments, the antibodies in the formulation are stable for at least 5 minutes, or at least 10 minutes, or at least 15 minutes, or at least 20 minutes, or at least 30 minutes, or at least 60 minutes, or at least 90 minutes, or at least 120 minutes after placement of the antibody product formulation in the mouth.

In one embodiment, the dose may be a whole soft-boiled egg. A soft-boiled egg can be enriched with the avian antibodies against the targeted immunogens. The soft-boiled egg can include avian antibodies that have not been inactivated in the cooking process and that can coat the mucous membranes of the individual.

In some embodiments, the formulations described herein can be formulations against SARS-CoV-2 to reduce and/or prevent Covid-19 in individuals. In some embodiments, avian antibodies can be generated in hens against viral antigens from SARS-CoV-2 as described above. In one embodiment, the avian antibodies generated against the SARS-CoV-2 antigens can inhibit the binding and attachment of SARS-CoV-2 to cellular attachment site, e.g. ACE2 receptor, in the individual. In some embodiments, tablets, candies, gummies, throat lozenges, nasal inhalants and the like are used to coat the pharyngeal areas in an individual to act as a mucosal protectant against infection from SARS-CoV-2.

In one embodiment, the present description can include a kit for nasal administration. The kit can include powdered egg material containing avian antibodies in a bag such as a plastic bag. The kit may also include a scoop or other similar device to scoop a desired dose from the bag when needed by the individual administering the dose. The kit may also include a straw for inhalation of the egg powder. When desired, the appropriate dose of the egg powder is removed from the bag and placed on a hard surface. The straw can be used to inhale the egg powder into the nasopharyngeal area to act as a mucosal protectant. The egg powder can be stable without refrigeration. The egg powder can be stable in ambient temperatures even in tropical climates. The kit described herein can be advantageous as a low-cost protection against respiratory disease throughout the world.

In one embodiment, the present description can include oral formulations that are packaged for distribution. The formulations can be individually wrapped with one piece of a product, e.g. individually wrapped candy. In some embodiments, multiple pieces can be packaged together, e.g. a daily supply or a convenient number of products to carry in a purse or a pocket.

The present description can include methods of inhibiting the ability of viruses to adhere to the mucous membranes in the pharyngeal area of humans to prevent respiratory infections and disease. The method can include administering a nasal and/or an oral formulation to coat the nasopharyngeal area and/or the oropharyngeal area in an individual. The administration may be self-administration and/or administration by others. In some embodiments, administration can include placing the oral formulations in the mouth of an individual and allowing the avian antibody material to disperse in the mouth and coat the oropharyngeal area to act as a mucosal protectant. In some embodiments, administration can include administration of nasal formulations into each of the nostrils to coat the nasopharyngeal area to act as a mucosal protectant. In some embodiments, the method can be used to inhibit SARS-CoV-2 that causes the respiratory disease Covid-19.

In one embodiment, the method may include administering avian antibody material against one virus. In one embodiment, the method may include administering avian antibody material against one or more strains of the virus. In one embodiment, the method can include administering avian antibody material against more than one virus. In one embodiment, the method can include administering avian antibody material against more than one strain of more than one virus.

The present description can include methods of reducing disease transmission. The method can bind and neutralize the viruses in the oral and nasal passages in an individual when the formulations are administered to the individual. The method can include administering a nasal and/or an oral formulation to coat the nasopharyngeal area and/or the oropharyngeal area in an individual. The administration may be by self-administration and/or administration by others. In some embodiments, administration can include placing the oral formulations in the mouth of an individual and allowing the avian antibody material to disperse in the mouth and coat the oropharyngeal area to act as a mucosal protectant. In some embodiments, administration can include administration of nasal formulations into each of the nostrils to coat the nasopharyngeal area to act as a mucosal protectant. In some embodiments, the method can be used to inhibit SARS-CoV-2 that causes the respiratory disease Covid-19.

The method can include intermittent administration or continuous administrations. In one embodiment, the method can include simultaneous administration of more than one piece of the formulation, e.g. two or more tablets, candies, gummies. In one embodiment, the method can include sequential administration of a piece of formulation. The pieces may be administered as needed depending on the level of protection needed. If entering a public setting with unknown infection status of the individuals, a continuous supply of product may be administered. Administration of the products may be reduced and/or eliminated if an individual is in a residential setting or other setting with reduced exposure to potential sources of infection.

In some embodiments, the antibody product may be placed in the mouth and chewed to release the antibody. In some embodiments, the antibody may be placed in the mouth and allowed to slowly dissolve over time to slowly release the avian antibodies. In one exemplary embodiment, a candy may be placed in the mouth and held underneath the tongue and/or the upper lip. Limiting the access of the formulation from the saliva can protect the formulation from saliva proteases. The formulations may be ingested in a manner that prolongs the residence time in the mouth for a slow release of the antibodies into the mouth from the formulation. Slow release of the antibodies can prolong the protection provided by the formulation. As shown below in Examples, the IgY antibodies in the formulation are stable when slowly released. In some embodiments, the antibodies in the formulation are stable for at least 5 minutes, or at least 10 minutes, or at least 15 minutes, or at least 20 minutes, or at least 30 minutes, or at least 60 minutes, or at least 90 minutes, or at least 120 minutes after placement of the antibody product formulation in the mouth.

The present description can include methods of reducing disease transmission by infected individuals. The method can bind and neutralize the viruses in the oral and nasal passages in an infected individual. This can result in reducing the number of infectious viral particles expelled by the infected individual into the environment in respiratory droplets, air particles and the like. The expelled particles may have reduced number of infectious viral particles since the viral particles may be bound and neutralized by the avian antibody material. The method can include administering a nasal and/or an oral formulation to coat the nasopharyngeal area and/or the oropharyngeal area in an infected individual.

In one embodiment, the method may include administering avian antibody material against one virus. In one embodiment, the method may include administering avian antibody material against one or more strains of the virus. In one embodiment, the method can include administering avian antibody material against more than one virus. In one embodiment, the method can include administering avian antibody material against more than one strain of more than one virus, e.g. multiple strains of multiple viruses.

The avian antibody material can be delivered several times a day, e.g. as a nasal formulation and/or an oral formulation, as described herein, depending on expected exposure to provide longer term or more intense protection. In order to protect an individual during viral outbreaks, the individual susceptible to virus exposure can carry formulations containing avian antibody material. Individuals may carry the formulations and self-administer the formulations multiple times in a day to decrease the chance of infection from a virus or viruses. Individuals could easily apply the formulations to their pharyngeal areas prior to expected exposure by the method of administering the formulations. These avian antibodies would serve as a mucosal protectant to prevent the adherence and replication of the targeted virus or other microorganism in the individual.

The avian antibodies against a virus may be prepared on a yearly or other periodic time frame as new strains of the virus emerge. The avian antibodies administered may be based on the circulating strains of the viruses. As circulating strains change or the threat of a new, pandemic virus appears; simply immunizing new hens with the newest strain or strains of interest may update the product. The birds may begin to deposit antibodies into their eggs specific to the newest strain or strains in as little as two weeks after immunization.

### EXAMPLES

### Example 1—Materials and Methods

Selection of Egg Laying Avian Hens-White Leghorns, 16-19 weeks old were injected with the adjuvanted spike protein-S1. The S1 spike protein expressed in HEK cells was purchased from Acro Biosystems, Newark, DE. S1 spike protein was used to inject hens. Each hen was injected with 3.75 micrograms of protein. The hens were injected twice about two weeks apart. Samples from eggs were collected 2 weeks following the last injection.

Analysis of Individual Eggs—Eggs were monitored monthly for antibody responses to the specific antigens. Selected chickens were monitored at day 0 and continued on a monthly basis after the fourth month. The whole egg was collected from the shell and then a 1 ml sample was taken. This sample was then extracted with buffer to analyze the antibody content. The standard ELISA's for the spike protein immunogen was used for analysis. The negative readings were subtracted from the OD readings. Samples of egg yolks, egg powders and candies were tested.

ELISA Tests—This procedure is for testing chicken IgY in samples that may be eggs, serum, etc., but may be adapted for different species or otherwise. The sample containing IgY was extracted by preparing a 1:20 (w/v or v/v) in EEB and allow to rock on a tilter for twenty minutes. Further antibody dilutions were prepared with BSA. A general starting point was a 1:600 dilution for spray dried preparations and purified IgY and a 1:300 for liquid samples. Dilutions were vortexed. 200 μl of sample was added to wells, according to plate diagram. The plates were incubated at 25 °C for 1 hour. After the incubation was complete, the plate was washed three times with a plate washer. A solution of 15 ml of conjugate diluent and 30 μl of conjugate was made and mixed. 100 μl of conjugate solution was added to every well, except 1A. The plates were incubated at 25 °C for 1 hour. The plates were washed four times with the plate washer once the incubation is complete. The developing solution was made. 7.5 ml of solution B was mixed with 7.5 ml substrate (2 part KPL Kit). 100 μl of TMB was added to every well and incubated 10 minutes at 25 °C. 100 μl of stop solution (1N H2SO4) was added to every well. The plates were read at 450 nm.

Products with specific IgY antibodies—A variety of products were made using the anti-S1 spike protein IgY antibodies from eggs derived from hens immunized by the S1 spike protein. The egg yolks were separated from the egg whites and shells. The processed egg yolks were used in formulations to make antibody products as described below. Antibody products such as candies, gummies, tablets were made. Processes for making the antibody products with the IgY antibodies from egg yolks included a variety of ingredients described below. The IgY antibodies were maintained at temperatures below about 160 °F (about 72 °C) during the processing steps to make the IgY antibody products.

Amounts of Anti-S1 spike protein antibody molecules in antibody products—The amount of specific egg yolk antibody molecules in a product can be determined as follows. The following calculations are for a product that includes about 0.3333 grams of yolk powder per piece, e.g. a tablet. The following calculations refer to a tablet but it will be understood that similar calculations can be used to determine the number antibody molecules in other antibody products such as candies, gummies, and the like.

There are about 100 milligram IgY antibodies/yolk. Of the 100 mg, 10 mg are specific anti-S1 antibodies are expected. The molecular weight of an IgY molecule is 180 kDA. A mole of IgY weighs about 180,000,000 milligrams. Thus, 10 mg of specific IgY antibody/180,000,000 mg in a mole of IgY antibody results in 0.00000005556 (fraction of a mole) of specific IgY antibody. Avogadro's number is 6.023×10²³ molecules/mole.

Thus, 0.00000005556×6.023×10²³=3.348×10¹⁶ of anti-S1 specific IgY molecules in one egg yolk.

On average, 1 egg yolk=5.9 grams solids

0.3333 grams yolk powder/piece was used.

5.9 g/yolk/0.3333 g powder/piece=17.7 tablets/yolk

3.348×10¹⁶/17.7=1.89×10¹⁵ anti-S1 spike protein specific IgY antibody molecules/piece.

Approximately, the piece produced have 1.89×10¹⁵ of anti-S1 spike protein specific IgY antibody molecules/piece.

### Example 2—Characterization of the IgY Antibodies

Testing samples of egg yolks for immunogen binding—Samples of egg yolks were tested for stability by binding the specific IgY antibodies to the immunogen using an ELISA test. The stability of the antibodies through the production processes was evaluated. A 1 gram sample was used to start the dilution process. The results are shown in Table 1.

TABLE 1

| Sample | Absorbance | Dilutions |
|---|---|---|
| BSA | 0.01 | |
| Norm | 0.02 | 1:600 |
| Positive | 2.66 | 1:600 |
| Egg Pool | 2.41 | 1:600 |
| Raw | 2.69 | 1:300 |
| Sample | 2.45 | 1:600 |
| | 1.83 | 1:1200 |
| | 1.42 | 1:2400 |
| Pasteurized | 2.27 | 1:300 |
| Sample | 2.35 | 1:600 |
| | 1.88 | 1:1200 |
| | 1.21 | 1:2400 |
| Dried | 2.35 | 1:300 |
| Sample | 2.29 | 1:600 |
| | 2.03 | 1:1200 |
| | 1.59 | 1:2400 |

As can be seen in Table 1, the raw, pasteurized and dried samples all have antibodies that specifically bind to the immunogen. This shows the IgY survived the thermal processed of pasteurization and spray drying.

Assessment of SARS-CoV-2 neutralization activity by Plaque Reduction Neutralization Assay (PRNA)—The chicken-derived purified IgY samples labeled 052120, 060820, 061520 and 062220 were assessed for their ability to neutralize SARS-CoV-2 virus using a PRNA assay. Briefly, the antibodies were diluted 1:10 in culture medium and incubated with 200 plaque forming units (PFUs) of the Washington Strain of SARS-CoV-2 (USA-WA1/2020). SARS-CoV-2 was diluted in supplemented DMEM to appropriate concentration. Virus was then added to antibody samples and allowed to incubate for 1 hour at 37 °C and 5% CO₂. After incubation, viral plaque assay was conducted to quantify viral titers. 12-well plates were previously seeded with Vero cells (ATCC CCL-81) at a density of 2E5 cells per well. Media was aspirated from plates and virus-antibody samples were transferred to wells, one sample per well. Plates were inoculated for 1 hour at 37 °C and 5% CO₂. After infection, a 1:1 overlay consisting of 0.6% agarose and 2× Eagle's Minimum Essential Medium without phenol red (Quality Biological, 115-073-101), supplemented with 10% fetal bovine serum (FBS) (Gibco, 10,437,028), non-essential amino acids (Gibco, 11140-050), 1 mM sodium pyruvate (Corning, 25-000-C1), 2 mM L-glutamine, 1% P/S was added to each well. Plates were incubated at 37 °C for 48 hours. Cells were fixed with 10% formaldehyde for 1 hour at room temperature. Formaldehyde was aspirated and the agarose overlay was removed. Cells were stained with crystal violet (1% CV w/v in a 20% ethanol solution). Viral titer of SARS-CoV-2 was determined by counting the number of plaques.

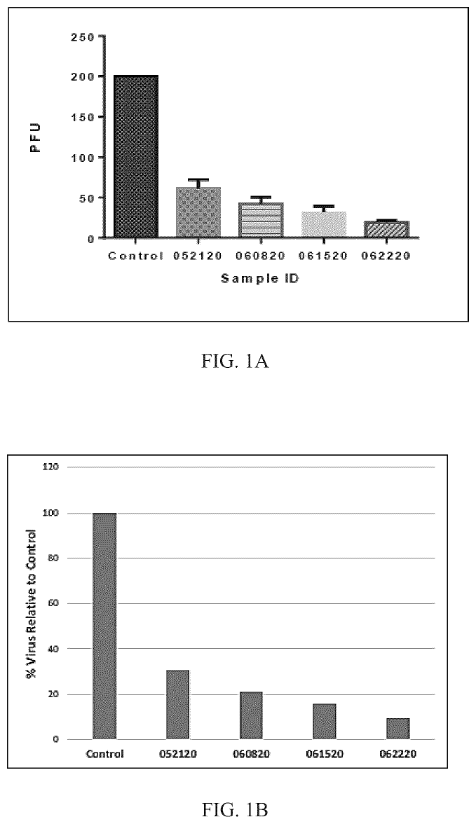

FIG. 1A depicts the absolute neutralization of virus observed in this assay at 1:10 dilution of the antibodies. Control refers to virus count in the absence of the antibody. FIG. 1B depicts the same data as relative percentage of neutralization observed in the context of the four antibodies as compared to the virus control. As shown in FIG. 1A and FIG. 1B, the IgY derived from chickens inoculated with the spike protein specifically bind to the SARS-CoV-2 virus.

### Example 3—IgY Antibody Products

Anti-Salmonella IgY Candies—Candies were made with commercially available egg yolk powder with anti-Salmonella antibodies to test the IgY antibody stability in candies. The candies included about 10% by weight of egg yolk powder. The stability of the antibodies in the antibody products was evaluated by extraction of the antibodies from the antibody product and evaluating the binding ability of the IgY antibody after extraction.

Extraction method: A 1:20 (w/v) of candy was placed in extraction buffer. The extraction buffer includes 5.8% NaCl and 1.7% K₂HPO₄ at a neutral pH. The candy was allowed to dissolve in the extraction buffer at 37 °C for 1 hour. 2 candy samples were tested.

Antibody Testing: After the candies dissolved in the extraction buffer, the antibody levels in the extraction buffer were tested using an anti-Salmonella ELISA, (an inhouse indirect ELISA). Absorbance values are shown in Table 2.

TABLE 2

| | Dilutions | Absorbance |
|---|---|---|
| Egg powder | 1:600 | 2.478 |
| | 1:1200 | 2.025 |
| Candy 1 | 1:30 | 1.747 |
| | 1:60 | 1.488 |
| | 1:120 | 1.207 |
| | 1:240 | 0.795 |
| | 1:480 | 0.489 |
| Candy 2 | 1:30 | 1.632 |
| | 1:60 | 1.119 |
| | 1:120 | 0.724 |
| | 1:240 | 0.448 |
| | 1:480 | 0.305 |

Extraction of the IgY from candy into the extraction buffer demonstrated that the IgY antibodies maintained their ability to bind the immunogen in the candy.

Anti-S1 spike protein candies—Candy were made with anti-S1 egg yolk. Candies were made with liquid egg yolk or dried egg yolk powder. Liquid Egg Recipe—1 cup pasteurized egg yolk was used. The egg yolk was separated and the removed from yolk sac. The egg yolk was heated at 62.2 °C for 6.2 minutes).

8 cups of gum paste mix (Wilton brand) was measured out. 1 Tablespoon of desired flavor and 10-12 drops of food coloring were added to the liquid egg yolks to form liquid egg yolk mix. The liquid egg yolk mix was added to the gum paste mix and mixed and/or kneaded. Additional paste mix was added if mixture is still sticky. The candy mixture was rolled out on a flat surface to a desired thickness with additional paste mix (if needed) and granulated sugar. A cutter was used to generate pieces that are about 1 gram in weight. Each batch made about 800 pieces.

Dry Egg Recipe with Pre-made Gum Paste—100 grams pre-made gum paste (Sunnyside brand), 20 grams powdered egg and 4 drops flavored oil were combined and mixed/kneaded. The candy mixture was rolled out on a surface and a cutter was used to generate pieces.

Dry Egg Recipe with Powdered Gum Paste—About 1 and ⅔ cups powdered gum paste (Sunnyside brand), ⅓ cup egg powder, 2 Tbs water and 4 drops flavored oil were combined and mixed/kneaded. The candy mixture was rolled out on a surface and a cutter was used to generate pieces.

Candy prepared with anti-S1 egg yolk was analyzed for the stability of the IgY in the candy. The IgY from the candy was extracted and tested for the ability to bind the spike protein.

Extraction methods: A solution 1:20 (w/v) of candy to extraction buffer was prepared. The extraction buffer was 5.8% NaCl and 1.7% K₂HPO₄ at a neutral pH. The candy was allowed to dissolve at room temperature for 1 hour on a rotating mixer.

Antibody Testing: The solution was tested using an anti-S1 ELISA, an in-house indirect ELISA. Absorbance Values are provided in Table 3.

TABLE 3

| | Dilution | Absorbance |
|---|---|---|
| Pos. Control | 1:600 | 2.768 |
| Candy 1 | 1:600 | 2.173 |
| | 1:1200 | 1.889 |
| Candy 2 | 1:600 | 1.641 |
| | 1:1200 | 1.641 |

Preparation of a Gummy product with anti-S1 spike protein antibodies—Egg yolk powder with anti-S1 antibodies was also used to make gummy, e.g. chewable, products. Table 4 below shows one formulation for a gummy product.

TABLE 4

| Ingredients | Amounts by wt. % |
|---|---|
| Water | 19.57% |
| Gelatin | 5.55% |
| Sweeteners | 49.92% |
| Egg yolk powder | 10.75% |
| oil | 11.86% |
| Preservatives, | 2.35% |
| flavor, color, etc. | |
| Total | 100% |

### Example 4—Use of IgY Antibody Products

Candies, e.g. mints, were made with IgY preparations. The IgY preparations were either a water extraction (WSPF) or a polyethylene glycol (PEG) extraction. The mints were placed in the mouth and consumed at different rates. Chewing the mints resulted in the fastest solubilization or release of the IgY antibodies into the saliva of the mouth. Mints were also held in the mouth for prolonged period of time by placing under the tongue or under the upper lip for slower release of the IgY antibodies from the mints. The stability of the IgY antibodies was tested at different time points for the different methods of ingestion as shown below.

Table 5 shows the stability of the antibody in the saliva using different IgY preparations (PEG and WSPF purifications) placed in saliva. The stability of IgY antibodies in mints were also tested in saliva.

TABLE 5

| Sample | Dilution | Adjusted A450 |
|---|---|---|
| BSA | — | 0.060 |
| α-SARS-CoV-2 | 1:600 | 3.274 |
| S1 WSPF | 1:1200 | 2.979 |
| | 1:2400 | 2.382 |
| Saliva Control | 1:300 | 0.052 |
| | 1:600 | 0.063 |
| | 1:1200 | 0.07 |
| | 1:2400 | 0.048 |
| Saliva + α-S1 | 1:300 | 2.754 |
| PEG IgY | 1:600 | 2.481 |
| | 1:1200 | 2.188 |
| | 1:2400 | 1.423 |
| Saliva + α-S1 | 1:300 | 2.614 |
| WSPF IgY | 1:600 | 2.546 |
| | 1:1200 | 2.122 |
| | 1:2400 | 1.462 |
| Saliva + α-S1 | 1:300 | 2.713 |
| Mint | 1:600 | 2.177 |
| | 1:1200 | 1.741 |
| | 1:2400 | 1.02 |
| α-S1 Mint | 1:300 | 2.924 |
| Control | 1:600 | 2.921 |
| | 1:1200 | 2.329 |
| | 1:2400 | 1.614 |

The data in Table 5 indicates that IgY antibodies can be stable in saliva for some time.

Table 6 that shows mint held in mouth vs chewed. The mint held in the mouth dissolved after 10 minutes.

TABLE 6

| | Time | | Adjusted |
|---|---|---|---|
| Sample | (min.) | Dilution | A450 |
| BSA | — | — | 0.083 |
| α-Normal PEG | — | 1:1200 | 0.109 |
| IgY | — | 1:2400 | 0.094 |
| α-S1 PEG IgY | — | 1:1200 | 2.986 |
| | — | 1:2400 | 2.630 |
| Pre-Mint Saliva | — | 1:50 | 0.030 |
| Control | — | 1:100 | 0.007 |
| Mint Held in | 0 | 1:50 | 0.736 |
| Mouth | 5 | | 2.597 |
| | 10 | | 1.929 |
| | 15 | | 0.136 |
| | 20 | | −0.001 |
| Mint Held in | 0 | 1:100 | 0.584 |
| Mouth | 5 | | 2.395 |
| | 10 | | 1.883 |
| | 15 | | 0.107 |
| | 20 | | 0.014 |
| Mint Chewed | 0 | 1:50 | 2.175 |
| | 5 | | 1.042 |
| | 10 | | 0.462 |
| | 15 | | 0.046 |
| | 20 | | 0.025 |
| Mint Chewed | 0 | 1:100 | 2.103 |
| | 5 | | 0.892 |
| | 10 | | 0.342 |
| | 15 | | 0.044 |
| | 20 | | 0.032 |

The data in Table 6 indicates that holding the mint in the mouth provides longer protection than when the mint is chewed. After 10 minutes, the chewed mint stability was lower than the mint held in the mouth.

Table 7 show data for a mint that was held in the mouth for 120 minutes.

TABLE 7

| | Time | | Adjusted |
|---|---|---|---|
| Sample | (min.) | Dilution | A450 |
| BSA | — | — | 0.120 |
| Saliva | 0 | 1:50 | 0.118 |
| | 15 | | 0.429 |
| | 30 | | 0.710 |
| | 45 | | 0.609 |
| | 60 | | 0.459 |
| | 75 | | 0.372 |
| | 90 | | No data |
| | 105 | | 0.587 |
| | 120 | | 0.873 |
| Saliva | 0 | 1:100 | 0.121 |
| | 15 | | 0.299 |
| | 30 | | 0.454 |
| | 45 | | 0.483 |
| | 60 | | 0.337 |
| | 75 | | 0.334 |
| | 90 | | 0.456 |
| | 105 | | 0.467 |
| | 120 | | 0.587 |

The data in Table 7 indicates that stable antibody is still present and released even after 120 minutes if the mint is held in the mouth.

The IgY antibodies in the mints are stable and release stable antibodies. The mints are effective for a longer period of time if they are held in the mouth for as long as possible and active antibody is released even after 120 minutes.

### Example 5

Reactivity of the anti-S1 antibodies against SARS-CoV2 variants. Variants were expressed in HEK cell lines. 96 well plates were coated with the proteins at 1 μg/ml concentration. An indirect ELISA procedure was used.

The data in Table 8 shows the ELISA values against the variants and demonstrates that the anti-S1 antibodies generated are effective in binding the SARS-CoV2 variants shown.

TABLE 8

| | RBD | | | S1 | | | | |
|---|---|---|---|---|---|---|---|---|
| | | South | | | South | | NTD | Coated at ug/ml, 200 ul |
| Egg dilutions | Brazil | African | Original | UK | African | Original | Brazil | per well |
| 1:600 | 0.98 | 1.03 | 0.98 | 2.52 | 2.35 | 2.45 | 1.07 | Eggs sent from MF. |
| 1:1200 | 0.72 | 0.55 | 0.61 | 2.08 | 1.86 | 1.71 | 0.68 | |
| 1:2400 | 0.40 | 0.35 | 0.29 | 1.40 | 1.26 | 1.01 | 0.34 | |
| 1:4800 | 0.21 | 0.20 | 0.14 | 0.66 | 0.58 | 0.55 | 0.22 | |

RBD-Receptor Binding Domain.
NTD-N-terminal domain
All samples in duplicate, negative wells subtracted

### Prophetic Example

Preparation of a Table with Anti-S1 Spike Protein Antibodies.

A 1-1.5 gram tablet can be made with 0.33 grams of hyperimmune egg yolk/tablet. Other ingredients will include compressible dextrose as a carrier/sweetener, color, flavoring and less than a half percent of an inert mineral lubricant, e.g. silicon dioxide to help release from the tablet press. The tablet press operates at 6 tons of pressure. There are many different types of presses and tablet sizes that can work. The machine that can be used is called a Stokes tablet press that makes about 150 tablets/minute. The flavors can be ginger, lemon, orange and honey. Other flavors may also be used. The tablets will be packaged in bulk and shipped. The tablets may be packaged in 20-30 tablet packages at the destination with a QR code.

Although the present invention has been described with reference to preferred embodiments, workers skilled in the art will recognize that changes may be made in form and detail without departing from the spirit and scope of the invention.

1.-21. (canceled)

22. A method of reducing SARS-CoV-2 viral load on a mucosal surface of an individual, comprising:
administering to a pharyngeal mucosal surface of the individual an oral formulation comprising avian IgY antibodies obtained from eggs laid by chickens inoculated with a recombinant SARS-CoV-2 S1 spike protein,
wherein the avian IgY antibodies are characterized by the ability to neutralize SARS-CoV-2 in a plaque reduction neutralization assay, and
wherein the formulation is administered in an amount effective to reduce viable SARS-CoV-2 present on the pharyngeal mucosal surface.

23. The method of claim 22, wherein the mucosal surface comprises an oropharyngeal mucosal surface, a nasopharyngeal mucosal surface, or both.

24. The method of claim 22, wherein the recombinant SARS-CoV-2 S1 spike protein is expressed in a recombinant expression system.

25. The method of claim 24, wherein the recombinant SARS-CoV-2 S1 spike protein is expressed in HEK cells.

26. The method of claim 22, wherein the avian IgY antibodies are directed against the recombinant SARS-CoV-2 S1 spike protein, a receptor binding domain (RBD) thereof, a N-terminal domain (NTD) thereof, or combinations thereof.

27. The method of claim 22, wherein the avian IgY antibodies bind a viral fusion molecule of SARS-CoV-2 and inhibit attachment of SARS-CoV-2 to an ACE2 receptor on epithelial cells of the pharyngeal mucosal surface.

28. The method of claim 22, wherein the plaque reduction neutralization assay comprises incubating SARS-CoV-2 with the avian IgY antibodies prior to infection of Vero cells.

29. The method of claim 22, wherein the avian IgY antibodies produce at least about 50% plaque reduction, optionally at least about 70% plaque reduction, at a 1:10 dilution in the plaque reduction neutralization assay.

30. The method of claim 22, wherein the oral formulation comprises whole egg material, egg yolk material, partially purified IgY antibodies, purified IgY antibodies, or combinations thereof.

31. The method of claim 30, wherein the partially purified IgY antibodies are obtained by water extraction or polyethylene glycol extraction of egg material.

32. The method of claim 30, wherein the oral formulation comprises spray-dried egg material.

33. The method of claim 32, wherein the spray drying is conducted at an outlet temperature below about 180 °F, optionally between about 150 °F and about 175 °F.

34. The method of claim 22, wherein the oral formulation is in the form of a tablet, throat lozenge, candy, or chewable gummy.

35. The method of claim 22, wherein the oral formulation comprises between about 0.1 grams and about 5 grams of egg material per administered dose.

36. The method of claim 22, wherein each administered unit of the oral formulation comprises between about 0.3 grams and about 0.7 grams of egg yolk powder and contains between about 1×10¹³ and about 1×10¹⁷ SARS-CoV-2-specific IgY antibody molecules, optionally about 1×10¹⁵ SARS-CoV-2-specific IgY antibody molecules.

37. The method of claim 22, wherein the oral formulation is held in the mouth for at least about 5 minutes, optionally at least about 10 minutes, 30 minutes, or 60 minutes, thereby providing detectable functional IgY antibodies in saliva for up to about 120 minutes.

38. The method of claim 22, wherein the oral formulation is administered at least twice daily, optionally at least three times daily, during a period of anticipated exposure to SARS-CoV-2.

39. The method of claim 22, wherein the recombinant SARS-CoV-2 S1 spike protein is derived from a SARS-CoV-2 variant selected from B.1.1.7, B.1.351, P.1, B.1.427, B.1.429, B.1.526, B.1.525, P.2, or combinations thereof.

40. The method of claim 22, wherein the oral formulation further comprises avian antibodies directed against at least one secondary respiratory pathogen selected from Streptococcus pneumoniae, Haemophilus influenzae, Staphylococcus aureus, Mycobacterium tuberculosis, or combinations thereof.

41. The method of claim 22, wherein the individual is infected with SARS-CoV-2 and administration of the oral formulation reduces expulsion of infectious viral particles, or wherein the individual is uninfected and administration of the oral formulation reduces risk of infection.

42. The method of claim 22, wherein the oral formulation further comprises a sweetener, gelatin, oil, preservative, flavoring, or combinations thereof.
